(19) United States
(12) Patent Application Publication
(10) Pub. No.: US 2005/0130252 A1
(43) Pub. Date: Jun. 16, 2005

(54) Stabilized susceptibility tests of aerobic pathogens

(75) Inventors: Patricia A. Bradford, New York, NY (US); Peter J. Petersen, Chester, NY (US)

Correspondence Address: WYETH;PATENT LAW GROUP, 5 GIRALDA FARMS, MADISON, NJ, 07940, US

(73) Assignee: Wyeth Holdings Corporation, Madison, NJ (US)

(21) Appl. No.: 11/010,240

(22) Filed: Dec. 10, 2004

Related U.S. Application Data
(60) Provisional application No. 60/572,209, filed on May 18, 2004.
(60) Provisional application No. 60/528,618, filed on Dec. 11, 2003.

(51) Int. Cl. C12Q 1/02

(52) U.S. Cl. 435/28

(57) ABSTRACT

The present invention is directed to the field of microbiology and in particular to compositions and methods for determining susceptibility of aerobic pathogens to antibiotics. The addition of adjuvants to the compositions of the invention stabilize the test medium and eliminates inconsistencies in susceptibility testing, most especially testing involving the tetracycline family of antibiotics.

This application claims priority from copending provisional application No. 60/528,618 filed on Dec. 11, 2003 and application No. 60/572,209 filed on May 18, 2004 the entire disclosures of which are hereby incorporated by reference.

### FIELD OF THE INVENTION

The present invention is directed to the field of microbiology and in particular to compositions and methods for determining susceptibility of aerobic pathogens to antibiotics. The compositions and methods of the invention are especially useful for susceptibility testing, including susceptibility testing of the tetracycline family of antibiotics and especially susceptibility testing involving the 7 and 9-substituted tetracyclines and most especially tigecycline (TGC).

### BACKGROUND OF THE INVENTION

During the development of an antibiotic, quality control (QC) ranges for susceptibility tests (National Committee for Clinical Laboratory Standards, 2003. Methods for Dilution Antimicrobial Susceptibility Tests for Bacteria That Grow Aerobically; Approved Standards: M7-A6, Sixth ed, vol. 23., National Committee for Clinical Laboratory Standards Wayne, Pa., National Committee for Clinical Laboratory Standards 2003. Methods for Dilution Antimicrobial Disk Susceptibility Tests; Approved Standards: M2-A8, Eighth ed, vol. 23. National Committee for Clinical Laboratory Standards, Wayne, Pa.) must be established which are then utilized by clinical microbiology laboratories to determine if patient test results are valid. These QC ranges are determined and then defined for each antibiotic using a selected panel of organisms recommended by the National Committee for Clinical Laboratory Standards (NCCLS).

The QC ranges are established through a process, referred to as M23 studies, which involve testing by multiple laboratories, using different lots and manufacturers of media with testing on multiple days (National Committee for Clinical Laboratory Standards 2003. Development of In Vitro Susceptibility Testing Criteria and Quality Control Parameters; Approved Guideline: M23-A2, Second ed, vol. 20 No. 7. National Committee for Clinical Laboratory Standards, Wayne, Pa.) Tigecycline is a glycylcycline antibiotic currently in clinical development and is a broad spectrum antibiotic with equivalent activity against susceptible and multidrug resistant organisms (Sum, P. E. and P. Petersen, Bioorganic and Medicinal Chemistry Letters 9, 1459-1462, 1999). Tigecycline as a broad-spectrum antibacterial agent has potent activity against many gram-positive and gram-negative clinically relevant pathogens including resistant pathogens such as MRSA, VRE, PRSP and ESBL-producing Enterobacteriaceae (Biedenbach D. J., M. L. Beach, and R. N. Jones, Diagnostic Microbiology and Infectious Disease 40:173-177, 2001; Cercenado, E., S. Cercenado, J. A. Gomez, and E. Bouza, J. Antimicrob. Chemother. 52:138-139, 2003; Milatovic, D., F.-J. Schmitz, J. Verhoef, and A. C. Fluit, Antimicrob. Agents Chemother. 47:400-404, 2003; and Petersen, P. J., N. V. Jacobus, W. J. Weiss, P. E. Sum, and R. T. Testa, Antimicrob. Agents Chemother. 43:738-744,1999). During studies to obtain consistent quality control ranges for the American Type Culture Collection (ATCC) quality control organisms when tested against tigecycline, inconsistent MIC (minimum inhibitory concentration) values were obtained.

The present invention outlines novel methods and compositions to provide stabilized, consistent susceptibility test values for the ATCC quality control organisms.

These and other embodiments and features of the invention will be apparent from the following summary and description of the invention and from the claims.

### SUMMARY OF THE INVENTION

During the development of tigecycline, several studies were undertaken to establish the QC ranges to be used for Minimum Inhibitory Concentration (MIC) testing. In the first study, a preliminary QC range was established for each of the recommended QC organisms during the years involved in pre-clinical development. As tigecycline developed toward phase 2 clinical studies, a well-controlled NCCLS M23 study was done. The results of the second study (first M23 study) showed QC ranges that were one to two dilutions lower than the ranges that had been established during the previous five years of preclinical experience. Following the first M23 study, the QC ranges that had been established were accepted. However, both research laboratory experience and the experience of clinical microbiology laboratories performing the microbiology testing for clinical trials had variations with the QC, wherein tigecycline was frequently out of range because the MICs were too high. Subsequently, a second M23 study was performed. The results from the second M23 study compared to the QC limits established in the research laboratory and the experience of the clinical laboratories. These QC limits became the accepted values for those researchers working with tigecycline. It was further noted by certain laboratories, that the MICs of tigecycline when tested against QC organisms still varied and produced low, out of range, values. These values corresponded with the lower ranges established by the first M23 study.

It was further noted that there was a discrepancy of tigecycline MICs determined in fresh Mueller Hinton broth (MHB) and MICs determined in aged MHBs. When microbroth dilution MIC tests were performed in Mueller Hinton broth (MHB) that was fresh (<1 week old), the MIC results corresponded with the lower ranges found in the first described M23 study. It is a preferred embodiment of the invention that when testing Tigecycline in broth microdilution tests that the medium is prepared fresh and no greater than 12 hours old and in the absence of adjuvants when the titer plate is formed. However, if the MHB were aged (>1 week old) the MIC results correlated with the higher results from the second described M23 study. When using pre-prepared media (always >1 week old), variability was not observed and was similar to the second M23 study.

Accordingly, a need in the art exists for an effective means of providing for standardized susceptibility test results for ATCC quality control organisms when tested against tigecycline.

The present invention provides to the art novel methods and compositions to provide standardized MIC values for tigecycline when using Mueller-Hinton II broth (MHB) medium.

Tigecycline is a glycylcycline and is from the tetracycline family and having substitution at the 7 and 9 positions. Tigecycline is further referred to as GAR-936.

The invention described herein outlines novel methods and compositions wherein the susceptibility of bacteria to antibiotics can be characterized. These methods and compositions enhance the effectiveness of antibiotics, especially the tetracycline antibiotics.

In an effort to control the variability in MIC values of antibiotics, the inventors surprisingly and unexpectedly found that the addition of an adjuvant to the media allowed for the standardization of the determined MICs. The inventors further surprisingly and unexpectedly found that the addition of an adjuvant to the media suppressed the formation of an early peak and allowed for the standardization of the determined MICs.

In particular, to control the variability in MIC values, the inventors further discovered that the addition of an adjuvant derived from the cytoplasmic membranes of microorganisms such as Escherichia coli, (E. coli), which act as an oxygen scavenging or reducing agent permitted the standardization of the determined MICs.

It is an embodiment of this invention to provide a medium composition, which provides standardized, consistent values for Susceptibility Test results for ATCC quality control organisms.

A further embodiment of the present invention is to provide a medium composition, which provides standardized, consistent values for Susceptibility Test results for ATCC quality control organisms when tested against tetracyclines.

An additional embodiment of the present invention is to provide a medium composition, which provides standardized, consistent values for the Susceptibility Test results for ATCC quality control organisms when tested against 7 and 9-substituted tetracyclines.

A further embodiment of the present invention is to provide a medium composition, which provides standardized, consistent values for the Susceptibility Test results for ATCC quality control organisms when tested against tigecycline.

The methods and associated composition of the present invention provide, in a broad aspect, for the standardization of the Susceptibility Test results for tetracyclines, and in particular for tetracyclines which have substitution at the 7 and 9 positions and specifically for tigecycline.

New tigecycline (TGC) containing compositions have been discovered. The present compositions provide for standardized Susceptibility Test results for ATCC quality control organisms when tested.

Compositions comprising tigecycline, media and an adjuvant as components are provided. The present composition provides for the standardized Susceptibility Test results for ATCC quality control organisms when tested.

The invention further comprises a kit for determining the susceptibility of a micoorganism, said kit comprising one or more antibiotics in close confinement or proximity with media and adjuvant.

The MICs for TGC were determined under various conditions by broth microdilution using NCCLS procedures. The MHB was stored at 2° C., room temperature, anaerobically and with the addition of Oxyrase® to determine the effect of various conditions. Time Kill kinetics was used to confirm MIC differences observed with the broth microdilution for aged and fresh media results.

When tested in freshly prepared media, TGC was 2 to 3 dilution more active against the reference strains compared to commercially prepared and aged (7 day old) media from powder (MICs 0.03-0.12 and 0.12-0.25 μg/ml, respectively). Aged medium stored under anaerobic conditions prior to testing performed similar to fresh media (MICs 0.03-0.12 μg/ml). The addition of Oxyrase®, resulted in MICs similar to fresh medium (MICs 0.06-0.25 μg/ml). Time kill kinetics demonstrated a significant (>3 log₁₀) difference in viable growth when TGC was tested in fresh vs aged media (FIG. 1).

These and other embodiments are provided for by the invention disclosed and claimed herein.

### BRIEF DESCRIPTION OF THE DRAWINGS

The following is a brief description of the drawings which are presented for the purposes of illustrating the invention and not for purposes of limiting the same.

FIG. 1 shows the antibacterial activity of Tigecycline (TGC) In fresh and aged Mueller-Hinton broth against E. coli ATCC 25922

FIG. 2 shows the HPLC analysis of adding Oxyrase® to the medium and suppression of early peak formation

FIG. 3 shows the HPLC analysis of adding Oxyrase® to fresh and aged media and suppression of early peak formation.

FIG. 4 shows the proton nuclear magnetic resonance spectrum of the early peak.

### DETAILED DESCRIPTION OF THE INVENTION

In the description that follows, a number of terms used in the pharmaceutical arts and in vitro susceptibility testing are utilized. In order to provide a clear and consistent understanding of the specification and claims, including the scope to be given such terms, the following definitions are provided.

Adjuvant means a substance which enhances the effectiveness of medical treatment and further means a substance that may or may not have antimicrobial activity in and of itself, but in combination with an antibiotic in a sufficient amount, acts to stabilize and enhance susceptibility testing. Adjuvants include those selected from the group cysteine, thioglycolate, ascorbic acid, pyruvate and catalase. The methods and compositions described herein also include the use of adjuvants derived from the cytoplasmic membranes of microorganisms such as E. coli which act as stabilizing agents. OXYRASE® is the trademark for the cytoplasmic membranes of microorganisms such as E. coli marketed by Oxyrase, Mansfield, Ohio 44901, further known herein as Oxyrase® or Oxyrase® Enzyme System. Osyrase® is also known as a biocatalytic oxygen reducing agent. Further, the adjuvants which are oxygen reducing agents and those that are derived from the cytoplasmic membranes of microorganisms such as E. coli which contains oxygen scavenging membrane fragments, can be used alone or in combination with other adjuvants.

Adjuvant effect means the increase in the effectiveness of the selected antibiotic in the presence of an adjuvant. This effect is shown by culturing a microorganism, such as, for example, an infectious agent, or clinical isolate, or viable extract thereof that is capable of growth, in vitro in the presence of efficacious levels of one or more antibiotics, with and without an adjuvant. The presence of the adjuvant increases the effectiveness of the antibiotic, specifically the tetracycline family of antibiotics and in particular 7 and 9 substituted tetracyclines and specifically tigecycline. Bacterial growth can be described in either qualitative or quantitative terms. An example of a qualitative result would simply indicate growth or no growth which may be determined visually with the unaided eye. An example of a quantitative result would compare days in culture versus an index of growth as understood in the art. Examples of growth indices include simple graded symbols (e.g., “−, .+−., 1+, 2+, 3+ and 4+”) and numerical indicators (e.g., 0 to 999) such as that produced by the BACTEC 12B culture system (Becton Dickinson, Cockeysville, Md., USA). The significant aspect of the adjuvant effect is that there is an observed change in the growth characteristics of the microorganism, such change revealing itself in the context of the susceptibility test of the invention.

Adjuvant susceptibility test means the use of an adjuvant in combination with a tetracycline antibiotic in an in vitro assay for the purpose of establishing a pattern of antibiotic susceptibility of a microorganism. In the adjuvant susceptibility test, a sample containing the microorganism, for example, a homogeneous population of microorganisms or a mixture of types/variants/isolates, etc. of microorganisms, is exposed to a composition comprising an antibiotic, an adjuvant and a medium and the susceptibility of the microorganism in the sample to said antibiotic is determined based upon the viability of the microorganism in the sample. The adjuvant susceptibility test is an embodiment of the methods of the invention. Such susceptibility testing can be accomplished in a standard solid or broth media including Mueller-Hinton agar or Mueller-Hinton broth as known in the art, or other equivalent media. Such culture format would necessarily be supplemented with the appropriate antibiotic(s) and adjuvant in the appropriate combinations and at the appropriate concentrations as discussed herein. The purpose of the susceptibility test is to determine Antimicrobial Susceptibility to tetracyclines, and in particular to 7 and 9 substituted tetracyclines and further specifically to tigecycline.

A microorganism, being susceptible means that the microorganism is deleteriously affected by an antibiotic in such a manner that such clinical isolate or infectious agent is rendered incompetent, noninfectious or non-viable as understood in the art (Yao, J. D. C. et al., In: Murray, P. R. et al., eds. Manual of Clinical Microbiology, ASM Press, Washington, D.C. (2003) pp. 1039-1073).

Susceptible, as used herein, is synonymous with susceptibility. When a microorganism, such as a clinical isolate or infectious agent, is determined to be susceptible to a given antibiotic, the antibiotic is said to have activity against, or be active against such isolate or infectious agent.

Susceptibility testing or test means an in vitro assay whereby the susceptibility of a microorganism, such as a clinical isolate or an infectious agent, to a series of antimicrobial compounds, in particular tetracyclines, including 7 and 9 substituted tetracyclines and specifically tigecycline is determined, as understood in the art (Jorgensen, J. H. et al., In: Murray, P. R et al., eds. Manual of Clinical Microbiology, ASM Press, Washington, D.C. (2003) pp. 1102-1107; Jorgensen, J. H. et al., In: Murray, P. R. et al, eds. Manual of Clinical Microbiology, ASM Press, Washington, D.C. (2003) pp. 1108-1127)). (National Committee for Clinical Laboratory Standards, 2003. Methods for Dilution Antimicrobial Susceptibility Tests for Bacteria That Grow Aerobically; Approved Standards: M7-A6, Sixth ed, vol. 23., National Committee for Clinical Laboratory Standards Wayne, Pa., National Committee for Clinical Laboratory Standards 2003. Methods for Dilution Antimicrobial Disk Susceptibility Tests; Approved Standards: M2-A8, Eighth ed, vol. 23. National Committee for Clinical Laboratory Standards, Wayne, Pa.) The goal of the susceptibility testing described herein is to more accurately determine Antimicrobial Susceptibility Test results for a tetracycline antibiotic including 7 and 9 substituted tetracyclines and specifically tigecycline.

Antibiotic means any of the compounds known in the art that have a deleterious effect on the viability, integrity, infectivity or competence of an infectious agent, as understood in the art (see: Yao, et al, In: Murray, P. R. et al., eds. Manual of Clinical Microbiology, ASM Press, Washington, D.C. (2003) pp. 1039-1073 and Inderlied, C. B. et al, In: Murray, P. R. et al., eds. Manual of Clinical Microbiology, ASM Press, Washington, D.C. (2003) pp 1149-1177)). Kucers, A. et al., The Use of Antibiotics 4.sup.th ed. J. B. Lippincott Co. Philadelphia, Pa. (1987); and Lorian, V. ed. Antibiotics in Laboratory Medicine 2.sup.nd Edition, Williams & Wilkins, Baltimore, Md.). In particular an important class of antibiotics are tetracyclines, including 7 and 9 substituted tetracyclines and specifically tigecycline. The term antibiotic is synonymous with antimicrobial, therapeutic, or drug as used herein. All antibiotics are drugs or therapeutics, but not all drugs or therapeutics are antibiotics.

Tetracycline or, in particular, the tetracycline family of antibiotics, as used herein, means an antibiotic having as a nucleus a hydronaphthacene structure as understood in the art (Yao, J. D. C. et al., In: Murray, P. R. et al., eds. Manual of Clinical Microbiology, ASM. Press, Washington, D.C. (2003) pp. 1051-1052)). Kucers, A. et al., The Use of Antibiotics 4.sup.th ed. J. B. Lippincott Co. Philadelphia, Pa. (1987) pp. 979-1044) and in particular the 7 and 9-substituted tetracyclines and glycylcyclines. Examples of tetracyclines that are useful in the methods of the invention include, but are not limited to, tetracycline, chlortetracycline, oxytetracycline, dimethylchlortetracycline, demeclocycline, methacycline, lymecycline, clomocycline, doxycycline, and minocycline. Examples of glycylcyclines that are useful in the methods of the invention include, but are not limited to N,N-dimethylglycylamido 9-aminominocycline (DMG-Mino), N,N-dimethylglycylamido 9 amino-6-demethyl-6-deoxytetracycline (DMG-DMDOT) and tigecycline. It is reasonably expected that tetracyclines and glycylcyclines with chemical structures homologous to any of the above named tetracycline or glycylcycline compounds will also be useful in the methods of the invention.

Clinical isolate means a purified strain of a bacterial agent that causes infection, such clinical isolate being derived from a patient infected with such infectious agent. One or more clinical isolates could be derived from the same patient, or the same isolate might be derived from different patients, such as is seen during nosocomial outbreaks (Pittet, D. et al., Archives of Internal Medicine 155:1177-1184, (1995)). Such clinical isolates are typically purified by a combination of specimen processing and culture methods. As such these clinical isolates are viable and, therefore, available for further analysis and testing with respect to susceptibility to antibiotics in an in vitro assay such as a susceptibility test. Procedures for purifying these clinical isolates include methods and procedures known in the art, especially those described by Kent, P. T. et al., “Public Health Mycobacteriology: A Guide for the Level III Laboratory”, U.S. Department of Health and Human Service, Centers for Disease Control, Atlanta, Ga. (1985) pp. 31-70, and for the isolation of Mycobacterium, or the methods outlined by Pfyffer, G. E. et al., In: Murray, P. R. et al., eds. Manual of Clinical Microbiology, ASM Press, Washington, D.C. (2003) pp. 532-559)) and Brown, J. M. et al., In: Murray, P. R. et al., eds. Manual of Clinical Microbiology, ASM Press, Washington, D.C. (2003) pp. 502-531)). for the isolation of Nocardia spp., and Funke, G. et al., In: Murray, P. R. et al., eds. Manual of Clinical Microbiology, ASM Press, Washington, D.C. (2003) pp. 472-501)). for the isolation of Coryneform gram positive rods.

Infectious agent means an infectious microorganism, especially an infectious bacterium as understood in the art. Infectious agents of special interest according to the methods of the invention include those that cause disease (Isenberg, H. D. et al., In: Murray, P. R. et al., eds. Manual of Clinical Microbiology, ASM Press, Washington, D.C. 1995 pp 5-18))). A human or animal patient having a disease caused by such an infectious agent is said to have an infection caused by such an agent, or to be infected with such agent. An infectious agent that causes disease is said to be pathogenic. Bacteria that are typically not pathogenic, and part of the patient's normal bacterial flora, are said to be commensal. Under some circumstances, such as when the patient is immune compromised or immune suppressed (e.g., being infected with HIV, or having AIDS complex, or after having undergone an organ transplant), such commensal microorganisms can cause infection. A patient can be infected with one or more infectious agents.

The minimum inhibitory concentration (MIC) is the lowest concentration of antimicrobial agent that completely inhibits growth of the organism in the microtiter wells as detected by the unaided eye (National Committee for Clinical Laboratory Standards, 2003. Methods for Dilution Antimicrobial Susceptibility Tests for Bacteria That Grow Aerobically; Approved Standards: M7-A6, Sixth ed, vol. 23., National Committee for Clinical Laboratory Standards Wayne, Pa.)

By exposing a sample containing a microorganism to a composition of the invention is intended by mixing the microorganism and the composition, or otherwise providing for contact between the microorganism and the composition.

Thus, in its broadest embodiment, the invention is directed to a method for characterizing the susceptibility of microorganisms to antimicrobial compounds. In an additional embodiment, the microorganism being tested is an infectious agent or clinical isolate. In a further embodiment, the microorganism to be tested is obtained from a sample taken from a patient suspected of being, or at risk of being, or identified as being infected with undesired bacteria. The susceptibility test of the invention is herein referred to as the adjuvant susceptibility test. The results of such susceptibility testing characterize the microorganisms under investigation that are present in a sample, such as a clinical isolate or infectious agent, with respect to the adjuvant effect described herein. That is, the adjuvant susceptibility test of the invention identifies whether or not a microorganism(s) that is present in a sample is susceptible to the antibiotic(s) that are tested. Preferably, as a result of the adjuvant susceptibility test of the invention, antibiotics, and in particular, tetracyclines including 7 and 9 substituted tetracyclines and specifically tigecycline are identified to which the microorganisms present in the sample are susceptible. However, it is also important that, as a result of the susceptibility test of the invention, wherein antibiotics, in particular, tetracyclines combined with adjuvant are identified to which the microorganisms present in the sample are not susceptible.

The present invention further relates to a liquid or solid medium composition for the stabilization of Susceptibility Test results for ATCC quality control organisms. It has been discovered that the addition of an adjuvant derived from the cytoplasmic membranes of microorganisms such as E. coli, to the medium, stabilizes the Susceptibility Test results for ATCC quality control organisms. While not being bound by theory, using an oxygen reducing agent as an adjuvant reduces the oxygen content of the medium and stabilizes in particular, the Susceptibility Test results for ATCC quality control organisms. In particular, the addition of an oxygen reducing agent as an adjuvant suppresses the formation of an early peak as determined by HPLC.

The medium composition comprises a nutrient medium, an adjuvant and the antibiotic of choice. Optionally, the adjuvant may be an oxygen reducing agent, including Oxyrase®. Additional adjuvants are selected from the group cysteine, thioglycolate, ascorbic acid, pyruvate and catalase in the range of about 0.0005% to about 5.0% (weight/volume of the medium composition [hereinafter referred to as w/v]), preferably in the range of about 0.005% to about 0.5% (w/v) and most preferably about 0.05% (w/v).

It has been found that the inclusion of an adjuvant as an oxygen reducing agent, and in particular oxygen reducing agents such as those derived from the cytoplasmic membranes of microorganisms such as E. coli allows for standardization of the determined Susceptibility Test results for ATCC quality control organisms when tested against, in particular 7 and 9 substituted tetracyclines. When said adjuvant is derived from the cytoplasmic membranes of microorganisms such as E. coli and in particular Oxyrase® in the range of about 0.5% to about 10.0% (volume/volume of the medium composition [hereinafter referred to as v/v]), preferably about 1.0% to about 4.0% (v/v) and most preferably about 2.0% (v/v).

The process for preparing susceptibility test medium are the following steps:
a. Preparing Mueller Hinton Broth II medium by adding 22 grams powdered media to 1.0 liter distilled water;
b. Autoclaving (121° C., 15 psi, 15 minutes) and cooling the medium;
c. Adding Oxyrase® up to about 10% final volume and keeping at 35-37° C. in an incubator for 30 minutes;
d. Adding about 50 μL to each well of a 96 well microtiter plate;
e. Weighing drug and adding broth to drug to bring up to about (standard 128 μg/ml);
f. Adding 50 μL of the drug to the first column of the titer plate;
g. Diluting by two-fold serial diluting across or down the plate.
h. Preparing a Prompt™ inoculum at 10⁸ CFU/ml;
i. Diluting inoculum 1:100 in the broth at 10 μL in 9.9 ml (1:100) is 10⁶ colony forming units (CFU) forming the adjusted inoculum;
j. Adding 50 μL of the adjusted inoculum to the wells of the microtiter plate;
k. Incubating the plates at 35-37° C. for 18-22 hours; and
l. Reading, with the unaided eye, the MIC.

The nutrient media utilized in the invention is any liquid or solid preparation suitable for susceptibility testing.

Preferably, the nutrient media described herein is especially useful for susceptibility testing, including susceptibility testing of the tetracycline family of antibiotics and especially susceptibility testing involving the 7 and 9-substituted tetracyclines and most especially tigecycline.

Solid medium usually consists of liquid medium which have been solidified (i.e. “gelled”) with an agent such as agar or gelatin. Examples of commonly available medium being suitable for use for stabilization of break points for ATCC quality control organisms in the present invention, include, but are not limited to, Brain Heart Infusion, Brucella, CDC Anaerobe, Nutrient, Schaedler, Thioglycollate, HTM (Haemophilus Test Medium) or Trypticase Soy. (Difco Manual 11 th Edition. 1998. Difco Laboratories Division of Becton Dickinson Company Sparks, Md.). These are in both broth or agar form and may be supplemented with blood for growth of fastidious organisms requiring additional nutrients.

The medium may be made anaerobic through the use of Oxyrase®. enzyme system available from Oxyrase, Inc. of Mansfield, Ohio. In this regard, “Oxyrase®. for Agar” is a filtered enzyme additive used to produce anaerobic conditions in a wide variety of bacteriological agar medium. Similarly, “Oxyrase®. for Broth” is an enzyme additive used to produce anaerobic environments in bacteriological broth medium. Both of these mediums (media) are commercially available in sterile (EC) and non-sterile (EC/NS)-form.

The above-identified Oxyrase® enzyme system consists of an enzyme system derived from the cytoplasmic membranes of microorganisms such as E. coli. The commercially available agents consists of a buffered suspension of membrane particles, 0.2 microns or smaller. The enzyme system is active over wide pH and temperature ranges. The exact amount of membranes containing the enzyme systems needed to reduce oxygen in the medium varies by a number of parameters including pH, temperature, kinds and amounts of substrate present, surface to depth ratio of the container, and headspace volume.

The preferred Oxyrase® enzyme system utilized as an adjuvant in the invention is comprised of oxygen scavenging membrane fragments which contain an electron transport system which reduces oxygen to water in the presence of a hydrogen donor. These oxygen scavenging membrane fragments can be derived from the cytoplasmic membranes of bacteria and/or from the membranes of mitochondrial organelles of a large number of higher non-bacterial organisms. Other known biocatalytic oxygen reducing agents such as glucose oxidase, alcohol oxidase, catalase, etc. can also be supplemented or utilized in the present invention, although generally less preferably.

The preferred Oxyrase® enzyme system suitable for use in the invention include the use of sterile membrane fragments derived from bacteria having membranes which contain an electron transport system which reduces oxygen to water in the presence of a hydrogen donor in the nutrient medium. It is known that a great number of bacteria have cytoplasmic membranes which contain the electron transport system that effectively reduces oxygen to water if a suitable hydrogen donor is present in the medium. Bacterial sources are selected from the group Escherichia coli, Salmonella typhimurium, Gluconobacter oxydans, and Pseudomonas aeruginosa. These bacterial membranes have been highly effective in removing oxygen from media and other aqueous and semi-solid environments.

The present invention is especially useful for determination of the Susceptibility Test results for ATCC quality control organisms and further for characterizing and testing clinical isolates or treating disease caused by infectious microorganisms.

The methods of the invention are directed to a method wherein a microorganism is tested for susceptibility to antibiotics. In an embodiment of the invention, the microorganism is a clinical isolate. In a preferred embodiment of the invention the microorganisms are ATCC quality control organisms. Such testing procedures or therapeutic regimes are useful for any desired microorganism, and especially, any desired bacterium.

The methods of the invention, especially for the susceptibility test, are also conveniently practiced by providing the agents used in such method in the form of a kit. Such a kit preferably contains appropriate growth media (liquid or solid) and adjuvants and test organisms as controls for the kit or combinations thereof, antibiotic(s) or combinations thereof, and if desired, water of the appropriate purity. The control organisms, adjuvants, media and/or antibiotics in the collection can be one that is not tailored for a particular microorganism, or one that is specifically tailored to a particular microorganism. Specific kits may, if desired, contain, inter alia, particular organisms to use, preferably, as standards. In such a kit, if the non-bacterial components are not already mixed together as they might be, such components are generally in close proximity to each other, even if confined in separate containers or packages, and in close proximity to any bacterial samples, which may optionally be ATCC quality control organisms, provided in the kit.

It will be understood by those with skill in the art that the invention may be performed within a wide and equivalent range of conditions, parameters and the like, without affecting the spirit or scope of the invention or any embodiment thereof. The following non-limiting examples illustrate certain aspects of the present invention.

Materials and Methods

Organisms:

Bacterial isolates used were those recommended by the NCCLS for quality control for susceptibility testing. (National Committee for Clinical Laboratory Standards, 2003. Methods for Dilution Antimicrobial Susceptibility Tests for Bacteria That Grow Aerobically; Approved Standards: M7-A6, Sixth ed, vol. 23., National Committee for Clinical Laboratory Standards Wayne, Pa., National Committee for Clinical Laboratory Standards 2003. Methods for Dilution Antimicrobial Disk Susceptibility Tests; Approved Standards: M2-A8, Eighth ed, vol. 23. National Committee for Clinical Laboratory Standards, Wayne, Pa.). Standards included E. coli ATCC 25922, S. aureus ATCC 29213, E. faecalis ATCC 29212, S. pneumoniae ATCC 49247 and H. influenzae ATCC 49247. Clinical isolates of E. coli, S. aureus, E. faecalis, S. pneumoniae, S. pyogenes, M. catarrhalis, and H. influenzae were obtained from the Wyeth General Culture Collection. E. coli and S. aureus strains expressing characterized tetracycline resistance determinants have been described previously (Petersen, P. J., N. V. Jacobus, W. J. Weiss, P. E. Sum, and R. T. Testa, Antimicrob. Agents Chemother. 43:738-744,1999).

Antibiotics and Chemicals.

A standard powder of tigecycline was obtained from Wyeth Research, Pearl River, N.Y. Minocycline, tetracycline, L-cysteine, L-ascorbic acid, sodium pyruvate, catalase and sodium thioglycolate were purchased from Sigma/Aldrich Chemical Company (St. Louis, Mo.) Oxyrase® was purchased from Oxyrase Inc, Mansfield, Ohio.

Susceptibility Testing.

The in vitro activities of tigecycline and control antibiotics were determined by the broth microdilution method as recommended by the NCCLS. (National Committee for Clinical Laboratory Standards, 2003. Methods for Dilution Antimicrobial Susceptibility Tests for Bacteria That Grow Aerobically; Approved Standards: M7-A6, Sixth ed, vol. 23., National Committee for Clinical Laboratory Standards Wayne, Pa.) was used for the standard procedures. MBH with 5% lysed horse blood was used for testing S. pneumoniae and Haemophilus Test Medium was used for testing H. influenzae. Oxyrase® (Oxyrase Inc, Mansfield, Ohio) was used according to the manufacturer's instructions by adding 1 ml of Oxyrase® to 50 ml of media. Media age studies were performed by preparing MHB in multiple flasks, then storing the media under various conditions. MICs were usually performed in duplicate on each day of testing. Storage under anaerobic conditions was conducted in an anaerobic chamber (MACS MG 1000, Don Whitley Scientific LTD). The pH of the media was determined by pH meter (Corning 430). A comparison of the in vitro activity of tigecycline by agar and broth dilution methodolgies with and without Oxyrase® against clinical isolates is shown in Table 1.

TABLE 1
Comparison of the In Vitro Activity of Tigecycline by Agar and Broth Dilution
Methodolgies with and Without Oxyrase ® Against Clinical Isolates

| | | | | Agar | | Broth | |
|---|---|---|---|---|---|---|---|
| | | | | Tigecycline | | Tigecycline | |
| | | | | Oxyrase ® | Agar | Oxyrase ® | Broth |
| | ORGANISM | ID # | IDENTIFIER | (10%) | Tigecycline | (2%) | Tigecycline |
| 1 | Escherichia coli | GC2270 | tet(M) | 0.12 | 0.25 | 0.12-0.06 | 0.50 |
| 2 | Escherichia coli | GC4559 | parent GC4560 | 0.12 | 0.25 | 0.12 | 0.50 |
| 3 | Escherichia coli | GC4560 | IMP mutant | 0.03 | 0.03 | 0.03 | 0.25 |
| 4 | Escherichia coli | GC2203 | ATCC Control | 0.12 | 0.25 | 0.12 | 0.25 |
| 5 | Escherichia coli | GC1073 | tet(B) wt | 0.25 | 0.25 | 0.12 | 0.50 |
| 6 | Staphylococcus aureus | GC1131 | Clinical isolate | 0.25 | 0.25 | 0.25 | 0.50 |
| 7 | Staphylococcus aureus | GC6466 | Clinical - tet(M) | 0.12 | 0.25 | 0.12 | 0.50 |
| 8 | Staphylococcus aureus | GC6467 | Clinical - tet(M) + (K) | 0.5 | 1 | 0.50 | 1 |
| 9 | Staphylococcus aureus | GC1079 | tet(K) | 0.25 | 0.5 | 0.25-0.12 | 0.50 |
| 10 | Staphylococcus aureus | GC4536 | Smith MP -In Vivo | 0.12 | 0.25 | 0.12 | 0.50 |
| 11 | Staphylococcus aureus | GC2216 | ATCC Control | 0.25 | 0.5 | 0.12 | 0.50 |
| 12 | Enterococcus faecalis | GC4555 | ATCC Control | 0.12 | 0.12 | 0.06 | 0.50 |
| 13 | Enterococcus faecalis | GC2267 | tet(L) + (M) + (S) | 0.12 | 0.25 | 0.12 | 0.50 |
| 14 | Enterococcus faecalis | GC2242 | Vancomycin- | 0.12 | 0.12 | 0.12 | 0.50 |
| | | | Resistant | | | | |
| 15 | Candida albicans | GC3066 | ATCC Control | >16 | >16 | >16 | >16 |

The effect of Oxyrase® on the in vitro activity (MIC, μg/ml) of tigecycline, other glycylcyclines and minocycline is presented in Table 2.

TABLE 2
Effect of Oxyrase ® on the In Vitro Activity (MIC, ug/ml)
of Tigecycline and other Glycylcyclines and Minocycline

| | | Tigecycline | DMG-DMDOT | DMG-MINO | Minocycline |
|---|---|---|---|---|---|
| | | Minimal Inhibitory Concentration (ug/ml) | | | |
| E. coli ATCC 25922 | 2% Oxyrase ® | 0.03 | 0.12 | 0.06 | 0.50 |
| S. aureus ATCC 25923 | 2% Oxyrase ® | 0.06 | 0.12 | 0.12 | 0.12 |
| E. faecalis ATCC 29212 | 2% Oxyrase ® | 0.03 | 0.06 | 0.03 | 2 |
| P. aeruginosa ATCC 27853 | 2% Oxyrase ® | 8 | 8 | 16 | 16 |
| E. coli ATCC 25922 | No Oxyrase ® | 0.12 | 0.12 | 0.25 | 1 |
| S. aureus ATCC 25923 | No Oxyrase ® | 0.25 | 0.25 | 0.50 | 0.25 |
| E. faecalis ATCC 29212 | No Oxyrase ® | 0.25 | 0.06 | 0.25 | 4 |
| P. aeruginosa ATCC 27853 | No Oxyrase ® | 16 | 16 | 16 | >16 |

Media Age vs Fresh Time-kill Experiment.

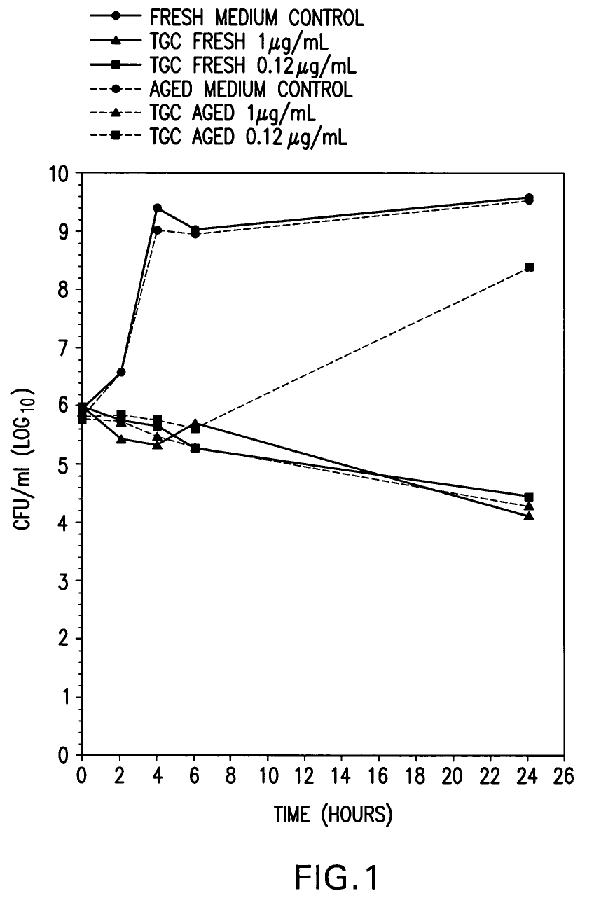

Time-kill assays were performed by the broth macrodilution method in accordance with the NCCLS guidelines (NCCLS. 1999. Methods for Determining Bactericidal Activity of Antimicrobial Agents; Approved Guidelines: M26-A, vol. 19. National Committee for Clinical Laboratory Standards, Wayne, Pa.). A starting inoculum of approximately 10⁶ CFU/ml and a final concentration of the antibiotic equal to four times the MIC were employed for these assays. Flasks containing 50 ml of MHB II with the appropriate antimicrobial agent were inoculated with 50 ml of the test organism in logarithmic growth phase. Test flasks (250 ml) were incubated with shaking (150 RPM) in a 35° C. water bath. Aliquots were removed for the determination of viable counts at 0, 2, 4, 6 and 24 hours. Serial dilutions were prepared in sterile 0.85% sodium chloride solution. The diluted samples (0.05 ml) were plated onto appropriate agar plates trypticase soy agar (TSA) with a spiral plater (Don Whitley Scientific LTD). The plates were incubated at 35° C. in ambient air for 18-22 hours and the number of colonies were determined on the ProtoCOL plate reader plater (Don Whitley Scientific LTD). Killing curves were constructed by plotting the log₁₀ CFU/ml versus time over 24 hours and the change in bacterial concentration was determined. Data is shown graphically in FIG. 1. Presented in Table 3 are the minimal inhibitory concentration results of the effect of media age, fresh vs. aged and Oxyrase® on the activity of tigecycline.

As shown in Table 3, the MICs of tigecycline determined in aged media that was supplemented with Oxyrase® were nearly identical to those determined in fresh media. Additionally, the MICs of tigecycline determined in fresh media that was also supplemented with Oxyrase® were not significantly different from the MICs determined in unsupplemented fresh media.

TABLE 3
Effect of Media Age and Oxyrase ® on the Activity of Tigecycline

| | | Freshly | Aged | Fresh |
|---|---|---|---|---|
| | | Prepared | Media + | Media + |
| | Aged | Media | Oxyrase ® | Oxyrase ® |
| Organism (N = 8) | Minimal Inhibitory Concentration (ug/ml) | | | |
| E. coli | 0.25-0.5 | 0.12-0.25 | 0.12-0.25 | 0.06-0.12 |
| ATCC 25922 | (0.25)* | (0.12) | (0.12) | (0.12) |
| S. aureus | 0.5 | 0.25 | 0.12-0.25 | 0.12-0.25 |
| ATCC 29213 | (0.5) | (0.25) | (0.25) | (0.12) |
| E. faecalis | 0.25-0.5 | 0.06-0.12 | 0.12 | 0.06 |
| ATCC29212 | (0.5) | (0.06-0.12) | (0.12) | (0.06) |

*(modal value)

Effect of Media Age on MICs of Tigecycline.

To determine the effect of media age on the in vitro activity of tigecycline, MHB was prepared and stored at room temperature or 2° C. for 28 days. Microbroth dilution MIC trays of tigecycline, minocycline and tetracycline were prepared on day 0, 7, 14, 21 and 28. MICs were determined in replicates of six on each day of testing as shown in Table 4. Further as shown in Table 4, there was a reproducible 3 to 4 dilution increase in the MICs of tigecycline over the four week period of testing. The aging of the media was slowed somewhat in the media stored at 4° C. compared to the media stored at room temperature.

As shown in Table 4, the effect of media age, preparation and storage on the in vitro activity (MIC, μg/ml) of Tigecycline against reference organisms ATCC 25922, ATCC29213 and ATCC29212 over the time interval of day 0 to day 28 are displayed.

TABLE 4
Effect of Media Age, Preparation and Storage on the In Vitro Activity
(MICs, ug/ml) of Tigecycline Against the ATCC Reference Organism

| | | |
|---|---|---|
| Fresh from | Fresh from | Pre- |
| Powder 2° C. | Powder^{a} | prepared^{a} |

E. Coli ATCC 25922 (N = 6)

| | | | |
|---|---|---|---|
| Day 0 | 0.03-0.06 | 0.03 | 0.12 |
| Day 1 | 0.03-0.06 | 0.06 | 0.12-0.25 |
| Day 7 | 0.06 | 0.12 | 0.12-0.25 |
| Day 14 | 0.12-0.25 | 0.12-0.25 | 0.12-0.25 |
| Day 21 | 0.25 | 0.12-0.25 | 0.5-0.25 |
| Day 28 | 0.12 | 0.12-0.25 | 0.12-0.25 |

S. aureus ATCC 29213 (N = 6)

| | | | |
|---|---|---|---|
| Day 0 | 0.12 | 0.06-0.12 | 0.25 |
| Day 1 | 0.12 | 0.12 | 0.25 |
| Day 7 | 0.12 | 0.25 | 0.25 |
| Day 14 | 0.25-0.5 | 0.25-0.5 | 0.25-0.5 |
| Day 21 | 0.25-0.5 | 0.25 | 0.50 |
| Day 28 | 0.25 | 0.25 | 0.25-0.5 |

E. faecalis ATCC 29212 (N = 6)

| | | | |
|---|---|---|---|
| Day 0 | 0.03 | 0.03 | 0.25 |
| Day 1 | 0.03 | 0.03-0.06 | 0.25 |
| Day 7 | 0.06 | 0.12-0.25 | 0.25 |
| Day 14 | 0.25 | 0.25 | 0.25-0.5 |
| Day 21 | 0.25 | 0.25 | 0.25-0.5 |
| Day 28 | 0.12-0.25 | 0.12-0.25 | 0.25-0.5 |

^{a}room temperature

TABLE 6
Effect of Media Source,
Age and Storage Conditions on the Activity of Tigecycline

| | Freshly | Commercially | Aged | Aged |
|---|---|---|---|---|
| | Prepared | Prepared | Aerobic | Anaerobic |
| Organism (N = 4) | Minimal Inhibitory Concentration (ug/ml) | | | |
| E. coli | 0.03 | 0.12-0.25 | 0.12-0.25 | 0.03-0.06 |
| ATCC 25922 | | | | |
| S. aureus | 0.06-0.12 | 0.25 | 0.25-0.5 | 0.06-0.12 |
| ATCC 29213 | | | | |
| E. faecalis | 0.03 | 0.12-0.25 | 0.25 | 0.03 |
| ATCC29212 | | | | |

As shown in Table 5, the tigecycline MICs determined in fresh media were 1 to 3 dilutions lower than those determined in aged media. To further establish this effect, a time kill experiment was performed with E. coli ATCC 25922 in fresh and aged media at 0.12 and 1 μg/ml of tigecycline, which represents 1×, and 4×MIC of this organism determined in aged media. As shown in FIG. 1, both 0.12 and 1 μg/ml of tigecycline suppressed the growth of E. coli ATCC25922 when tested in fresh media. However, when the testing was performed in aged media, there was regrowth of the strain after 6 h exposure to 0.12 μg/ml of tigecycline. With further reference to Table 5, to determine if the effect of the aged media was restricted to the QC organisms, MICs were determined in broth that was freshly prepared and compared to those determined in broth that had been stored for two weeks at room temperature (RT) using a panel of organisms that included both clinical isolates and strains expressing various tetracycline resistance determinants.

As there was no change in the pH of the media over time, this could not account for the discrepancies in the MICs of tigecycline determined in fresh media compared to those determined in aged media. Therefore, while not being bound by theory, it postulated that the cause of the discrepancy between the fresh and aged media may be due to acceleration of oxidative degradation, and the formation of an early peak, caused by an increase in the amount of dissolved oxygen in the broth media that occurs over time during the storage. Because it appeared that the concentration of dissolved oxygen in the broth medium was effecting the tigecycline MICs, a study was done to assess the effect of aging on media that has been stored in an anaerobic chamber. As shown in Table 6, media that was commercially prepared and media that had been aged 2 weeks at RT under room air resulted in tigecycline MICs that were 1-4 dilutions higher than the freshly prepared media. In contrast, media that had been stored under anaerobic conditions resulted in MICs of tigecycline that were nearly identical to the results obtained in fresh media.

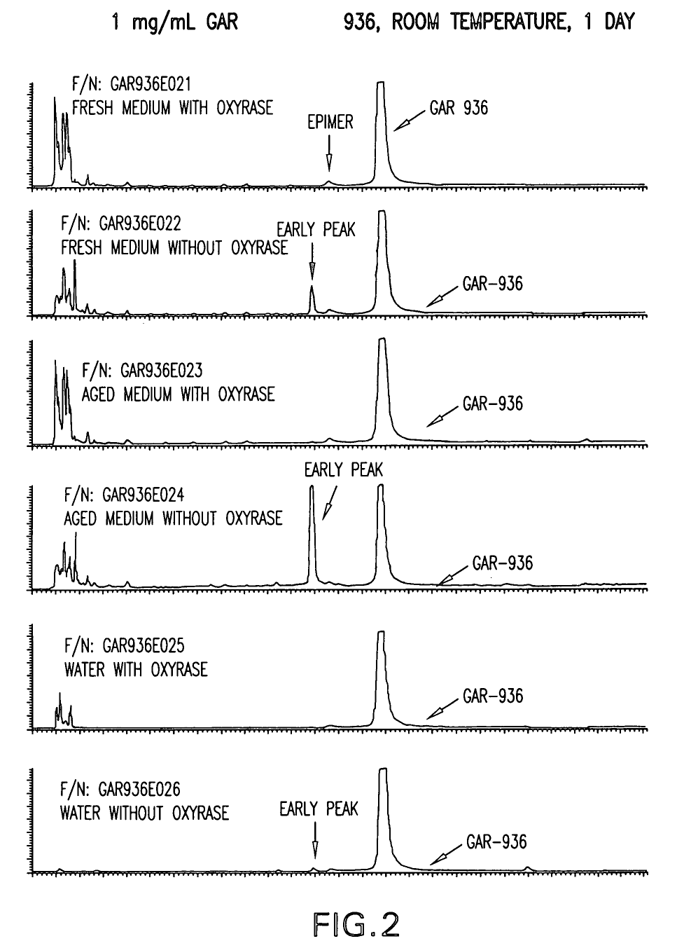

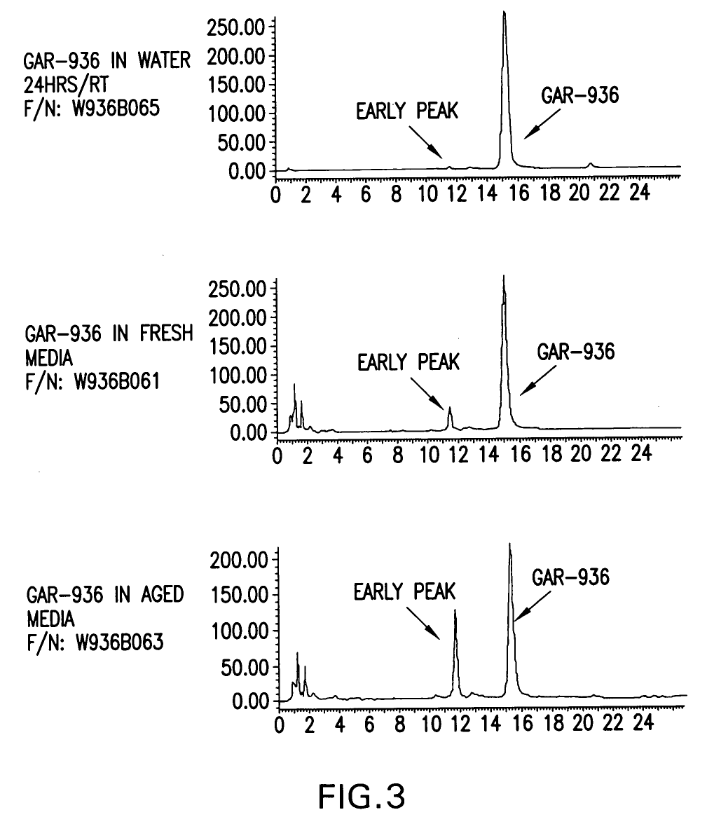

To test the hypothesis, solutions of tigecycline were prepared in water, fresh MHB and aged MHB. The solutions were stored overnight at RT, then subjected to HPLC analysis to look for degradation of tigecycline. As shown in FIG. 2, the HPLC analysis showed the early peak eluted at a retention time of about 11.5 to 12.0 minutes. As shown in FIG. 3, the amount of the early peak in fresh media was about 12 times the amount in water, whereas in aged media the ratio was about 35. This confirmed that the formation of early peak of tigecycline was accelerated in the aged media.

High Pressure Liquid Chromatography (HPLC).

In experiment 1 three experimental test samples were examined for early peak formation of tigecycline. In experiment 2 six experimental test samples were evaluated to determine the early peak of tigecycline which occurs in 24 hours. Results are as listed below:

| Experiment 1 vehicles: | Experiment 2 vehicles: |
|---|---|
| 1. fresh Mueller Hinton II | 1. fresh Mueller Hinton II |
| 2. aged Mueller Hinton II | with 2% Oxyrase ® |
| 3. water only | 2. fresh Mueller Hinton II |
| 4. aged Mueller Hinton II | without Oxyrase ® |
| without Oxyrase ® | 3. aged Mueller Hinton II |
| 5. water with 2% Oxyrase ® | with 2% Oxyrase ® |
| 6. water only | |

Tigecycline at a concentration of 1 mg/mL in each of the above vehicles was prepared, assayed at time zero and 1 day @ RT.

The HPLC parameters are listed below:
HPLC column: Luna C18(2), 5 μm, 4.6×150 mm
Detection: UV @ 250 nm
Flow Rate: 1.5 mL/min
Injection volume=30 μL
Mobile Phase A: 6.8 g KH2PO4 in 950 mL water, pH to 6.2 with KOH, mixed with 50 mL acetonitrile
Mobile Phase B: 6.8 g KH2PO4 in 500 mL water, pH to 6.2 with KOH, mixed with 500 mL acetonitrile

Gradient:

| Time (min) | % A | % B |
|---|---|---|
| 0 | 95 | 5 baseline |
| 20 | 60 | 40 linear |
| 5 | 0 | 100 linear |
| 1 | 0 | 100 hold |
| 0.1 | 95 | 5 baseline |

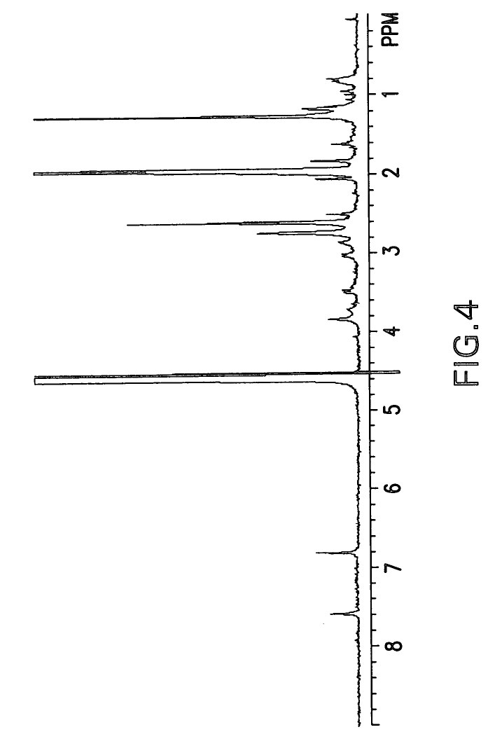

It is believed that the early peak has the following structural formula A with a characteristic proton nuclear magnetic resonance spectrum as shown in FIG. 4.

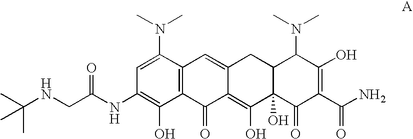

Structural formula A may also exist in tautomeric forms and the tautomers are depicted below:

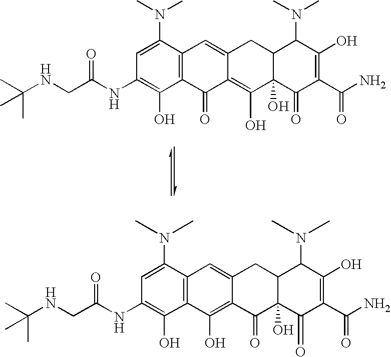

TABLE 5
Effect of Media Age on the in Vitro Activity
of Tigecycline Against Clinical Isolates

| | | Minimal Inhibitory | |
|---|---|---|---|
| | | Concentration (ug/ml) | |
| | | Fresh Media | Aged Media |
| | | Tigecycline | Tigecycline |
| 1 | E. coli GC2270 (tet(M)) | 0.06 | 0.25 |
| 2 | E. coli GC4559 (parent GC4560) | 0.06 | 0.12 |
| 3 | E. coli GC4560 (IMP mutant) | <0.008 | 0.06 |
| 4 | E. coli GC2203 (ATCC Control) | 0.03 | 0.12 |
| 5 | E. coli GC1073 (tet(B)) | 0.12 | 0.12 |
| 6 | S. aureus GC1131 (Clinical) | 0.25 | 0.25 |
| 7 | S. aureus GC6466 (tet(M)) | 0.12 | 0.25 |
| 8 | S. aureus GC6467 (tet(M) + (K)) | 0.50 | 0.50 |
| 9 | S. aureus GC1079 (tet(K)) | 0.25 | 0.25 |
| 10 | S. aureus GC4536 (Smith MP - | 0.12 | 0.25 |
| | In Vivo) | | |
| 11 | S. aureus GC2216 (ATCC Control) | 0.12 | 0.25 |
| 12 | E. faecalis GC4555 (ATCC Control) | 0.03 | 0.25 |
| 13 | E. faecalis GC2267 (tet(L) + (M) + (S)) | 0.12 | 0.25 |
| 14 | E. faecalis GC2242 (Van-resistant) | 0.06 | 0.12 |
| 15 | S. pneumoniae GC4465 (Clinical) | <0.008 | 0.03 |
| 16 | S. pneumoniae GC1894 (Clinical) | <0.008 | 0.03 |
| 17 | S. pyogenes GC4563 (Clinical) | <0.008 | 0.03 |
| 18 | M. catarrhalis GC6907 (Clinical) | 0.015 | 0.03 |
| 19 | H. influenzae GC6896 (ATCC Control) | 0.25 | 0.50 |
| 20 | C. albicans GC3066 ATCC (Control) | >8 | >8 |

To determine if the concentration of dissolved oxygen in the broth media could be controlled by the addition of a reducing agent to the medium, MICs were determined in the presence of 0.05% (w/v) L-cysteine, 0.05% L-ascorbic acid, 0.05% sodium pyruvate, 0.05% catalase and 0.05% sodium thioglycolate, all of which have been used previously reported as reducing agents in growth media for bacterial pathogens. The effect of reducing agents on the in vitro activity of tigecycline, minocycline and tetracycline is displayed in Table 7.

Tables 8 and 9 summarize the effect of Oxyrase® versus NCCLS Quality Control Strains—data combined from many independent experiments from nine investigators.

TABLE 7
Effect of Various Reducing Agents on the in Vitro Activity of Tigecycline,
Minocycline and Tetracycline

| | | Minimal Inhibitory Concentration (ug/ml) | | |
|---|---|---|---|---|
| Organism | Medium | Tigecycline | Minocycline | Tetracycline |
| E. coli ATCC 25922 | Mueller Hinton Broth II | 0.12-0.25 | 0.5-1 | 1-2 |
| S. aureus ATCC 29213 | (MHB II) | 0.25-0.5 | 0.25-0.5 | 0.5-1 |
| E. faecalis ATCC 29212 | | 0.5-0.25 | 4-8 | 16->16 |
| E. coli ATCC 25922 | Mueller Hinton Broth II | 0.06-0.25 | 0.5-1 | 1-2 |
| S. aureus ATCC 29213 | (Fresh) | 0.25 | 0.12-0.5 | 1-2 |
| E. faecalis ATCC 29212 | | 0.06-0.12 | 4 | >4 |
| E. coli ATCC 25922 | MHB II + | 0.06 | 0.5 | 1 |
| S. aureus ATCC 29213 | 2% Oxyrase ® | 0.12 | 0.12-0.25 | 0.5-1 |
| E. faecalis ATCC 29212 | | 0.03-0.06 | 2-4 | 16->16 |
| E. coli ATCC 25922 | MHB II + | 0.12 | 1 | 4 |
| S. aureus ATCC 29213 | 0.05% Thioglycolate | 0.25 | 0.25 | 1 |
| E. faecalis ATCC 29212 | | 0.12 | 8 | >16 |
| E. coli ATCC 25922 | MHB II + | 0.5 | 2 | 4 |
| S. aureus ATCC 29213 | 0.05% L-cysteine | 0.25 | 0.25 | 1 |
| E. faecalis ATCC 29212 | | 0.12 | 4 | >4 |
| E. coli ATCC 25922 | MHB II + | 2 | 4 | >4 |
| S. aureus ATCC 29213 | 0.05% L-ascorbic Acid | 2 | 1 | 2 |
| E. faecalis ATCC 29212 | | 1 | 4 | >4 |
| E. coli ATCC 25922 | MHB II + | 0.12 | 0.50 | 2 |
| S. aureus ATCC 25923 | 0.05% Sodium Pyruvate | 0.25 | 0.25 | 1 |
| E. faecalis ATCC 29212 | | 0.25 | 2 | 16 |
| E. coli ATCC 25922 | MHB II + | 0.50 | 1 | 4 |
| S. aureus ATCC 29213 | 0.05% Catalase | 0.50 | 0.50 | 1 |
| E. faecalis ATCC 29212 | | 0.50 | 4 | 16 |

TABLE 8
Distribution of MIC (ug/ml) for Tigecycline against the ATCC Quality Control
Strains a-j using Mueller Hinton Broth or Mueller Hinton Broth with Oxyrase ®

| | (n = 320)^{a} | (n = 240)^{b} | (n = 320)^{c} | (n = 240)^{d} | (n = 320)^{e} | (n = 240)^{f} |
|---|---|---|---|---|---|---|
| MIC (ug/ml) | Number of Occurrences | | | | | |
| 0.03 | | | | | | 5 |
| 0.06 | | 130 | | 14 | 1 | 203 |
| 0.12 | 24 | 103 | 1 | 203 | 96 | 28 |
| 0.25 | 210 | 5 | 219 | 22 | 140 | 2 |
| 0.5 | 23 | | 29 | 1 | 16 | 1 |
| 1 | 58 | 2 | 62 | | 64 | 1 |
| 2 | 4 | | 9 | | 3 | |
| 4 | 1 | | | | | |

| | | | |
|---|---|---|---|
| (n = 320)^{g} | (n = 240)^{h} | (n = 210)ⁱ | (n = 240)^{j} |

| | |
|---|---|
| MIC (ug/ml) | Number of Occurrences |

| | | | | |
|---|---|---|---|---|
| 0.004 | | 6 | | |
| 0.008 | | 73 | | 2 |
| 0.016 | 1 | 137 | | 1 |
| 0.03 | 54 | 21 | | |
| 0.06 | 188 | 3 | | 4 |
| 0.12 | 48 | | 13 | 156 |
| 0.25 | 29 | | 156 | 75 |
| 0.5 | | | 25 | 2 |
| 1 | | | 10 | |
| 2 | | | 6 | |

^{a}E. coli ATCC 25922 Mueller Hinton Broth
^{b}E. coli ATCC 25922 Mueller Hinton Broth + Oxyrase ®
^{c}S. aureus ATCC 29213 Mueller Hinton Broth
^{d}S. aureus ATCC 29213 Mueller Hinton Broth + Oxyrase ®
E. faecalis ATCC 29212 Mueller Hinton Broth
^{f}E. faecalis ATCC 29212 Mueller Hinton Broth + Oxyrase ®
^{g}S. pneumoniae ATCC 49619 Mueller Hinton Broth
^{h}S. pneumoniae ATCC 49619 Mueller Hinton Broth + Oxyrase ®
ⁱH. influenzae ATCC 49247 Mueller Hinton Broth
^{j}H. influenzae ATCC 49247 Mueller Hinton Broth + Oxyrase ®

TABLE 9
Distribution of MIC (ug/ml) for Tigecycline against ATCC Quality Control
Strains using Mueller Hinton Broth or Mueller Hinton Broth with Oxyrase ®

| | (n = 90)^{A} | (n = 33)^{B} | (n = 131)^{C} | (n = 29)^{D} | (n = 96)^{E} | (n = 31)^{F} |
|---|---|---|---|---|---|---|
| MIC (ug/ml) | Number of Occurrences | | | | | |
| 0.03 | | 3 | | | | 6 |
| 0.06 | 1 | 13 | 1 | 2 | | 14 |
| 0.12 | 15 | 14 | 27 | 15 | 25 | 11 |
| 0.25 | 47 | 3 | 78 | 12 | 52 | |
| 0.5 | 27 | | 25 | | 19 | |

^{A}E. coli ATCC 25922 Mueller Hinton Broth
^{B}E. coli ATCC 25922 Mueller Hinton Broth + Oxyrase ®
^{C}S. aureus ATCC 29213 Mueller Hinton Broth
^{D}S. aureus ATCC 29213 Mueller Hinton Broth + Oxyrase ®
^{E}faecalis ATCC 29212 Mueller Hinton Broth
^{F}E. faecalis ATCC 29212 Mueller Hinton Broth + Oxyrase ®

1. A medium composition for the determination of susceptibility testing for ATCC quality control organisms wherein said medium composition comprises a nutrient medium, an antibiotic and an adjuvant in a sufficient amount to stabilize and enhance susceptibility testing.

2. The medium composition of claim 1, wherein the adjuvant is selected from the group cysteine, thioglycolate, ascorbic acid, pyruvate and catalase.

3. The medium composition of claim 1, wherein the adjuvant is derived from the cytoplasmic membranes of microorganisms.

4. The medium composition of claim 1, wherein the antibiotic is a tetracycline.

5. The medium compositon of claim 1, wherein the antibiotic is a 7,9-substituted tetracycline.

6. The medium composition of claim 4 wherein the tetracycline antibiotic is selected from the group tetracycline, chlortetracycline, oxytetracycline, dimethylchlortetracycline, demeclocycline, methacycline, lymecycline, clomocycline, doxycycline, minocycline, N,N-dimethylglcylamido 9-aminominocycline (DMG-mino), N,N-dimethylglcylamido 9 amino-6-demethyl-6-deoxytetracycline (DMG-DMDOT) and tigecycline.

7. The medium composition of claim 6, wherein the tetracycline antibiotic is tigecycline.

8. The medium composition of claim 3, wherein the microorganisms are selected from the group Escherichia coli, Salmonella typhimurium, Gluconobacter oxydans, and Pseudomonas aeruginosa.

9. The medium composition according to claim 8 wherein the microorganism is Escherichia coli.

10. The medium composition of claim 9, wherein the adjuvant is present in the amount from about 0.5% to about 10.0% volume/volume of the medium composition.

11. The medium composition according to claim 10 wherein the adjuvant is present in the amount of about 1.0% to about 4.0% (v/v).

12. The medium composition according to claim 11 wherein the adjuvant is present in about 2.0% (v/v).

13. A medium composition for the determination of susceptibility testing for ATCC quality control organisms wherein said medium composition comprises a nutrient medium, a tetracycline antibiotic and an adjuvant in a sufficient amount to stabilize and enhance susceptibility testing.

14. The medium composition of claim 13, wherein the adjuvant is derived from the cytoplasmic membranes of microorganisms.

15. The medium composition of claim 13 wherein the tetracycline antibiotic is a 7,9-substituted tetracycline.

16. The medium composition of claim 13 wherein the tetracycline antibiotic is selected from the group tetracycline, chlortetracycline, oxytetracycline, dimethylchlortetracycline, demeclocycline, methacycline, lymecycline, clomocycline, doxycycline, minocycline, N,N-dimethylglcylamido 9-aminominocycline (DMG-mino), N,N-dimethylglcylamido 9 amino-6-demethyl-6-deoxytetracycline (DMG-DMDOT) and tigecycline.

17. The medium composition of claim 16, wherein the tetracycline antibiotic is tigecycline.

18. The medium composition of claim 14, wherein the microorganisms are selected from the group Escherichia coli, Salmonella typhimurium, Gluconobacter oxydans, and Pseudomonas aeruginosa.

19. The medium composition according to claim 18 wherein the microorganism is Escherichia coli.

20. The medium composition of claim 19, wherein the adjuvant is present in the amount from about 0.5% to about 10.0% volume/volume of the medium composition.

21. The medium composition according to claim 20 wherein the adjuvant is present in the amount of about 1.0% to about 4.0% in medium (v/v).

22. The medium composition according to claim 21 wherein the adjuvant is present in about 2.0% (v/v).

23. A method to stabilize minimum inhibitory concentration (MIC) test values for ATCC quality control organisms comprising the following steps:
a. adding to a medium an adjuvant in a sufficient amount to stabilize and enhance susceptibility testing and an antibiotic forming a medium for testing;
b. adding a test organism to said medium for testing, forming a test medium;
c. incubating said test medium; and
d. reading the MIC value.

24. The method according to claim 23, wherein the adjuvant is derived from the cytoplasmic membranes of microorganisms.

25. The method according to claim 23, wherein the antibiotic is a tetracycline.

26. The method according to claim 23, wherein the antibiotic is a 7,9-substituted tetracycline.

27. The method according to claim 23 wherein the tetracycline antibiotic is selected from the group tetracycline, chlortetracycline, oxytetracycline, dimethylchlortetracycline, demeclocycline, methacycline, lymecycline, clomocycline, doxycycline, minocycline, N,N-dimethylglcylamido 9-aminominocycline (DMG-mino), N,N-dimethylglcylamido 9 amino-6-demethyl-6-deoxytetracycline (DMG-DMDOT) and tigecycline.

28. The method according to claim 27, wherein the tetracycline antibiotic is tigecycline.

29. The method according to claim 24, wherein the microorganisms are selected from the group Escherichia coli, Salmonella typhimurium, Gluconobacter oxydans, and Pseudomonas aeruginosa.

30. The method according to claim 29 wherein the microorganism is Escherichia coli.

31. The method according to claim 30, wherein the adjuvant is present in the amount from about 0.5% to about 10.0% volume/volume of the medium composition.

32. The method according to claim 31 wherein the adjuvant is present in the amount of about 1.0% to about 4.0% in medium (v/v).

33. The method according to claim 32 wherein the adjuvant is present in about 2.0% (v/v).

34. A method to stabilize minimum inhibitory concentration (MIC) test values for ATCC quality control organisms comprising the steps:
a. Preparing Mueller Hinton Broth II medium by adding 22 grams powdered media to 1.0 liter distilled water;
b. Autoclaving (121° C., 15 psi, 15 minutes) and cooling the medium;
c. Adding an adjuvant up to about 10% final volume and keeping at 35-37° C. in an incubator for 30 minutes;
d. Adding about 50 μL to each well of a 96 well microtiter plate;
e. Weighing drug and adding broth to drug to bring up to about (standard 128 μg/ml);
f. Adding 50 μL of the drug to the first column of the titer plate;
g. Diluting by two-fold serial diluting across or down the plate.
h. Preparing an inoculum at 10⁸ CFU/ml;
i. Diluting inoculum 1:100 in the broth at 10 μL in 9.9 ml (1:100) is 10⁶ colony forming units (CFU) forming the adjusted inoculum;
j. Adding 50 μL of the adjusted inoculum to the wells of the microtiter plate;
k. Incubating the plates at 35-37° C. for 18-22 hours; and
l. Reading, with the unaided eye, the MIC.

35. The method according to claim 34, wherein the adjuvant is derived from the cytoplasmic membranes of microorganisms.

36. The method according to claim 34, wherein the drug is a tetracycline.

37. The method according to claim 34, wherein the drug is a 7,9-substituted tetracycline.

38. The method according to claim 36 wherein the tetracycline antibiotic is selected from the group tetracycline, chlortetracycline, oxytetracycline, dimethylchlortetracycline, demeclocycline, methacycline, lymecycline, clomocycline, doxycycline, minocycline, N,N-dimethylglcylamido 9-aminominocycline (DMG-mino), N,N-dimethylglcylamido 9 amino-6-demethyl-6-deoxytetracycline (DMG-DMDOT) and tigecycline.

39. The method according to claim 36, wherein the tetracycline antibiotic is tigecycline.

40. The method according to claim 35, wherein the microorganisms are selected from the group Escherichia coli, Salmonella typhimurium, Gluconobacter oxydans, and Pseudomonas aeruginosa.

41. The method according to claim 40, wherein the microorganism is Escherichia coli.

42. The method according to claim 41, wherein the adjuvant is present in the amount from about 0.5% to about 10.0% volume/volume of the medium composition.

43. The method according to claim 42 wherein the adjuvant is present in the amount of about 1.0% to about 4.0% in medium (v/v).

44. The method according to claim 43 wherein the adjuvant is present in about 2.0% (v/v).

45. A medium composition for the determination of susceptibility testing for ATCC quality control organisms wherein said medium composition comprises a nutrient medium, an adjuvant and an antibiotic wherein said adjuvant is present in an amount sufficient to suppress the formation of an early peak as determined by high pressure liquid chromatography with a retention time of about 11.5-12.0 minutes.

46. The medium composition of claim 45, wherein the adjuvant is derived from the cytoplasmic membranes of microorganisms.

47. The medium composition of claim 45, wherein the antibiotic is a tetracycline.

48. The medium compositon of claim 45, wherein the antibiotic is a 7,9-substituted tetracycline.

49. The medium composition of claim 47 wherein the tetracycline antibiotic is selected from the group tetracycline, chlortetracycline, oxytetracycline, dimethylchlortetracycline, demeclocycline, methacycline, lymecycline, clomocycline, doxycycline, minocycline, N,N-dimethylglcylamido 9-aminominocycline (DMG-mino), N,N-dimethylglcylamido 9 amino-6-demethyl-6-deoxytetracycline (DMG-DMDOT) and tigecycline.

50. The medium composition of claim 49, wherein the tetracycline antibiotic is tigecycline.

51. The medium composition of claim 46, wherein the microorganisms are selected from the group Escherichia coli, Salmonella typhimurium, Gluconobacter oxydans, and Pseudomonas aeruginosa.

52. The medium composition according to claim 51 wherein the microorganism is Escherichia coli.

53. The medium composition of claim 52, wherein the adjuvant is present in the amount from about 0.5% to about 10.0% volume/volume of the medium composition.

54. The medium composition according to claim 53 wherein the adjuvant is present in the amount of about 1.0% to about 4.0% in medium (v/v).

55. The medium composition according to claim 54 wherein the adjuvant is present in about 2.0% (v/v).

56. A medium composition for the determination of susceptibility testing for ATCC quality control organisms wherein said medium composition comprises a nutrient medium, an adjuvant and a tetracycline antibiotic wherein said adjuvant is present in an amount sufficient to suppress the formation of an early peak as determined by high pressure liquid chromatography.

57. A medium composition for the determination of susceptibility testing for ATCC quality control organisms wherein said medium composition comprises a nutrient medium, an adjuvant and a 7,9-substituted tetracycline antibiotic wherein said adjuvant is present in an amount sufficient to suppress the formation of an early peak as determined by high pressure liquid chromatography.

58. A medium composition for the determination of susceptibility testing for ATCC quality control organisms wherein said medium composition comprises a nutrient medium, an adjuvant and tigecycline antibiotic wherein said adjuvant is present in an amount sufficient to suppress the formation of an early peak as determined by high pressure liquid chromatography with a retention time of about 11.5-12.0 minutes.

59. The medium composition according to claim 2 wherein the adjuvant is present in the range of about 0.0005% to about 5.0% weight/volume of the medium composition.

60. The medium composition according to claim 59 wherein the adjuvant is present in the range of about 0.005% to about 0.5% (w/v).

61. The medium composition according to claim 60 wherein the adjuvant is present in about 0.05% (w/v).

62. A medium composition for the determination of susceptibility testing for ATCC quality control organisms wherein said medium composition comprises a fresh nutrient medium and an antibiotic to stabilize and enhance susceptibility testing.

63. The medium composition according to claim 62, wherein the fresh nutrient medium is no greater than 12 hours old.

64. The medium composition according to claim 62, wherein the antibiotic is tigecycline.
